(19) United States
(12) Patent Application Publication
(10) Pub. No.: US 2013/0217773 A1
(43) Pub. Date: Aug. 22, 2013

(54) Method and Device for assistance in delivery of medication, and Formulas

(71) Applicant: Mikhail Levitin, Reeders, PA (US)

(72) Inventor: Mikhail Levitin, Reeders, PA (US)

(73) Assignee: Mr. Mikhail Levitin, Reeders, PA (US)

(21) Appl. No.: 13/401,846

(22) Filed: Feb. 22, 2012

(51) Int. Cl. A61K 31/195; A61K 31/19; A61P 19/00; A61K 31/135; A61P 19/02; A61K 31/16; A61K 31/09

(52) U.S. Cl. 514/567; 514/626; 514/570; 514/561; 514/718; 514/654; 514/627

(57) ABSTRACT

This invention is designed for the convenient and reliable delivery of predetermined doses of topical therapeutic medications applied to the skin.

The importance of this method of application is that it allows reducing the amount of a therapeutic agent used in one session or reducing amount of sessions per day by eliminating the application of medications to a treating surface by hand.

The emphasis is on making therapeutic agents available for self-administration, especially for partially disabled people with conditions such as arthritis and/or visual impairments who would not be able to use these medications without assistance.

### DESCRIPTION OF PREFERRED EMBODIMENTS

The present invention relates generally to methods of, and devices for, assistance in administration of therapeutic agents covered by proposed formulas for achieving better therapeutic results with fewer side effects from the presented therapeutic agents.

The above-mentioned general types are shown in the art.

### SUMMARY OF THE INVENTION

It is an object of the present invention to provide a method of, and an apparatus for, assistance in applying a therapeutic agent to the targeted zone on a patient's body with fewer side effects and less amount or frequency of the applications due to elimination of an additional carrier such as a hand.

More particularly, it is a goal of the present invention to provide a portable, light and easily administered device for assistance in applying therapeutic agents to a target zone by using an applicator with a pump or a spray.

In keeping with these objects and with others which will become apparent hereinafter, one feature of the present invention is a method of applying a therapeutic agent to the skin with better control over the dose of the medication and use of a lesser amount of medication than has been commonly used on the market today.

The novel features which are characteristics of the present invention, are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a schematic drawing of a pumping device to deliver a therapeutic agent from a distance.

FIG. 2 is a schematic drawing of the inventive device for the applications of medications in forms of a gel, a cream, or an ointment.

### DESCRIPTION OF PREFERRED EMBODIMENTS

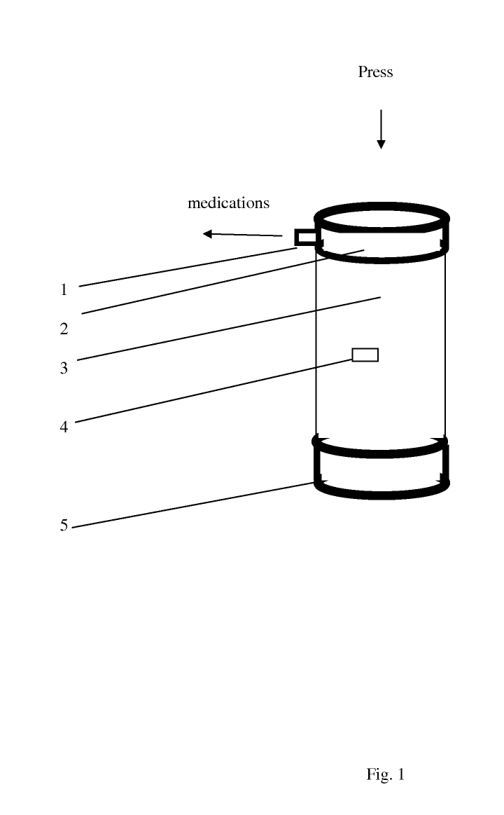

As shown in FIG. 1, a device that provides assistance in delivering a liquid medication to a target zone by pressing on a pump 2 of the device to spray through the supply 1 of the medication that is stored in the container 3 with the delivered amount registered in the counter 4 of the device and massaged onto the skin of the target area by an applicator 5 or said device.

In accordance with the present invention, the method of the delivery of the medications does not require any hand contact to provide an application.

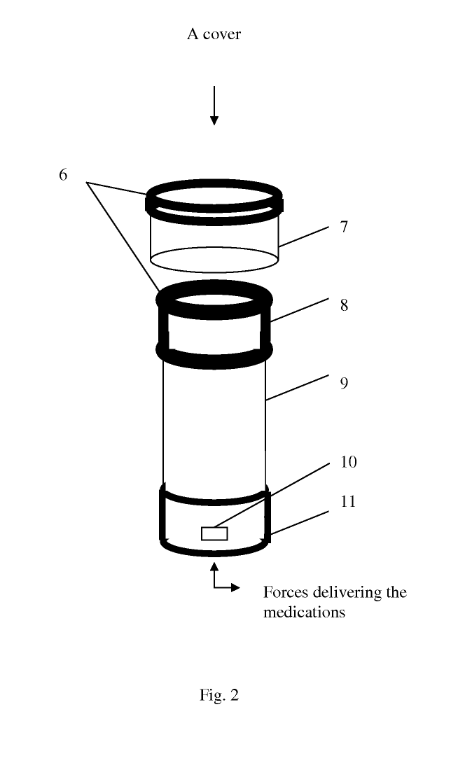

As shown in FIG. 2, a device that provides assistance in applications of therapeutic agents in form of a gel, a cream, or an ointment by rotating the delivery shuttle 11 clock wise to push the medication from the storage chamber 9 to the accumulating chamber 8 that will contain the necessary amount of medication calculated by the counter 10 and is ready for the direct application of the medication to the target zone from the chamber 8 rubbing in by the applicator 6 placed on said chamber 8 or on the cover 7.

It will be understood that each of the elements described above, or two or more together, may also find a useful application in other types, methods and constructions differing from the types described above.

When the invention has been illustrated and described as embodied in a method of, and a device for assistance in application of medications, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters of Patent is set forth in the appended claims.

I claim:

1. A spray like self operating by one hand drag distributing device delivering a liquid medication stored in a storage chamber of said device on a desirable surface of the body distanced from said device by pressing on a pump of said device to spray a liquid therapeutic agent such as Diclofenac sodium topical solution that is stored in said chamber of the device and allowing to make one discharge of the medication with one pressure/spray that is regulated by an air pressure in a controller of said device.

2. The method of claim 1 wherein the therapeutic agent is selected from the group consisting of Diclofenac sodium topical solution, Lidocaine HCL, Ketoprofen, Gabapentin Baclofen, Hudrophung Guafenesin, Nortriptylin, Capsaisin, Ethoxy Diglycol Polox.

3. The method of claim 1 wherein the therapeutic agent is present in an amount of Camphor 0.01-1.5%%, Menthol. 0.01-5.0%%, Lidocaine HCL 1.0 to 10.0%%, Diclofenac sodium topical solution from 1.0 to 10.0%%, Ketoprofen 1.0 to 10.0%%, Gabapentin 1.0 to 10.0%%, Baclofen 1.0 to 5.0%%, Hydromorphone HCl 1.0 to 2.5%%, Guafenesin 1.0 to 5.0%%, Capsaisin 1.0 to 69.0%%, Nortriptylin 0.1 to 1.0%%, and Ethoxy Diglycol Polox 10.0 to 30%%.

4. The method of claim 1 wherein the patient suffers from osteoarthritis of joints amenable to topical treatment, such as the knees and those of the hands, or pain due to neuropathy.

5. The method of claim 2 wherein the second therapeutic agent comprises up to 50 wt % of diclofenac sodium topical solution from 1.5 to 3.0%%.

6. A topical composition comprising a therapeutically effective amount of at least one therapeutic agent for treating a musculoskeletal condition, such as Ketoprofen, Gabapentin Baclofen, Diclofenac, Hudrophung Guafenesin, Lipoil, Capsaisin, Nortriptylin, Lipoil, Ethoxy Diglycol Polox, from about 0.0001 to 50 wt % of an anti inflammatory agent and optimally, a penetrating and/or hydrating agent and/or an ionotropic agent, application of the composition to the skin for treating said condition by releasing the therapeutic agent to the applied area.

7. The combination of claim 6 wherein said composition is formulated as a cream, gel, lotion spray, form, film, tape, ointment, plaster, or adhesive for application to skin.

8. The composition of claim 7, wherein said composition is directly delivered to the treating skin from a one hand self operating device without rubbing it by hand.

9. A device for assistance in applications of predetermined and countable doses of topical therapeutic medications described in claim 8.

10. The method of claim 9 wherein the patient suffers from osteoarthritis of joints amenable to topical treatment, such as the knees and those of the hands, or pain due to neuropathy.

11. A device described in claim 1 with a counter system as a structural part of said device that is preset for certain amount of a therapeutic agent delivered with each. Pump.

12. A device described in claim 1 with an applicator as a part of said device to provide massage, reflexology or acupressure combined with a therapeutic agent delivered on the skin with each pump from said device using the medication as a lubricant for said additional therapeutic modalities to increase the efficacy of the therapy.

13. The method of applying a therapeutic agent to a target zone without a contact with skin by the device described in claim 1 pressing the pump of said device with one or more fingers of holding said device hand.

14. A device for an assistance in the applications of predetermined and countable doses of topical therapeutic agents in form a cream, a gel, a lotion, form, film, tape, ointment, plaster, or adhesive, for example Guafenesin or Capsaisin, for application to skin by rotating the delivery shuttle of said device clock wise to push the medication from the storage chamber of the device to the accumulating chamber of the device providing the necessary amount of medication calculated by said counter of said device delivering the medication to the target zone from said chamber rubbing in by the applicator placed on said device or on the cover of the device.

15. A device described in claim 14 with a counter system of said device that has been preset for certain amount of a therapeutic agent delivered to an accumulating chamber of the device with each pump or each rotation clock wise of the delivery shuttle of the device to push the medication from the storage chamber to the accumulating chamber to reach desirable therapeutic effect without over dose the patient.

16. The method of applying a therapeutic agent to a target zone by the device described in claim 15 by pumping or rotating the delivery shuttle clock wise to push the medication from the storage chamber to the accumulating chamber delivering said medication on an applicator of said device to the targeting zone while boosting the therapeutic effect from the medication by using said applicator for massage, reflexology and acupressure to the targeting zone on the skin without a hand contact.
